(19) United States
(12) Patent Application Publication
(10) Pub. No.: US 2024/0238166 A1
(43) Pub. Date: Jul. 18, 2024

(54) TRICALCIUM SILICATE KIT AND METHOD FOR PREPARING PORTLAND CEMENT-BASED DENTAL MATERIAL USING THE SAME

(71) Applicant: Ming-Kuan Biomedical Technology Corporation, Xinfeng Township (TW)

(72) Inventors: Yung-Ming YANG, Xinfeng Township (TW); Kuan-Wei LU, Xinfeng Township (TW); Chih-Chung HUANG, Xinfeng Township (TW); Wei-Ling GAO, Xinfeng Township (TW); Yen-Tzu Lin, Xinfeng Township (TW); An-Cheng SUN, Xinfeng Township (TW)

(21) Appl. No.: 18/413,563

(22) Filed: Jan. 16, 2024

(30) Foreign Application Priority Data
Jan. 17, 2023 (TW) 112101931

(51) Int. Cl. A61K 6/851

(52) U.S. Cl. CPC A61K 6/851

(57) ABSTRACT

A tricalcium silicate kit includes a first reagent containing tricalcium silicate, and a second reagent containing a salt material and water. The salt material is selected from the group consisting of sodium carbonate, calcium chloride, and a combination thereof. A method for preparing a Portland cement-based dental material using the tricalcium silicate kit is also provided.

### CROSS-REFERENCE TO RELATED APPLICATION

This application claims priority to Taiwanese Invention Patent Application No. 112101931, filed on Jan. 17, 2023, which is incorporated by reference herein in its entirety.

### FIELD

The present disclosure relates to a tricalcium silicate kit and a method for preparing a Portland cement-based dental material using the tricalcium silicate kit.

### BACKGROUND

Ceramic materials, such as dental cements and dental bone materials which are commonly used on the market and which contain tricalcium silicate (3CaO·SiO₂, C₃S) as one of the main components, are often utilized in clinical dental treatments. The applicants have developed a method for preparing tricalcium silicate in a previous study, and the thus prepared tricalcium silicate can be directly mixed with pure water so as to allow the tricalcium silicate to solidify into a robust form in approximately 12 minutes to 14 minutes, and such solidified tricalcium silicate can be subsequently used in clinical dental treatments.

In spite of the aforesaid, there is still a need to develop a tricalcium silicate kit that can be utilized for preparing a Portland cement-based dental material.

### SUMMARY

Therefore, in a first aspect, the present disclosure provides a tricalcium silicate kit, which can alleviate at least one of the drawbacks of the prior art. The tricalcium silicate kit includes a first reagent containing tricalcium silicate, and a second reagent containing a salt material and water. The salt material is present in an amount ranging from 5 wt % to 45 wt %, based on the total weight of the second reagent. The salt material is selected from the group consisting of sodium carbonate, calcium chloride, and a combination thereof.

In a second aspect, the present disclosure provides a method for preparing a Portland cement-based dental material, which can alleviate at least one of the drawbacks of the prior art. The method includes mixing the first reagent with the second reagent of the aforesaid tricalcium silicate kit to undergo a hardening process, so as to form the Portland cement-based dental material. A ratio of a volume of the second reagent to a weight of the first reagent in decimal form ranges from 0.2 μL/g to 0.8 μL/g.

In a third aspect, the present disclosure provides another tricalcium silicate kit, which can alleviate at least one of the drawbacks of the prior art. The tricalcium silicate kit includes a first reagent containing calcium carbonate and tricalcium silicate, and a second reagent containing water. The calcium carbonate is present in an amount ranging from 2 wt % to 45 wt %, based on the total weight of the first reagent.

In a fourth aspect, the present disclosure provides another method for preparing a Portland cement-based dental material, which can alleviate at least one of the drawbacks of the prior art. The method includes mixing the first reagent with the second reagent of the aforesaid another tricalcium silicate kit to undergo a hardening process, so as to form the Portland cement-based dental material. A ratio of a volume of the second reagent to a weight of the first reagent in decimal form ranges from 0.2 μL/g to 0.7 μL/g.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

### First Embodiment of Tricalcium Silicate Kit

A first embodiment of a tricalcium silicate kit according to the present disclosure includes a first reagent containing tricalcium silicate, and a second reagent containing a salt material and water.

According to the present disclosure, the first reagent is packaged separately from the second reagent, and the tricalcium silicate kit does not include calcium carbonate.

According to the present disclosure, the first reagent may be pure tricalcium silicate or may further include an auxiliary agent (serving as a thickening agent) in addition to the tricalcium silicate, such that the first reagent is in a thick or paste-like form. In certain embodiments, the auxiliary agent is selected from the group consisting of ethanol, glycerol, polyethylene glycol (PEG) in liquid form, and combinations thereof. In certain embodiments, the PEG in liquid form has a molecular weight not greater than 600, such as polyethylene glycol 400 (PEG 400) or polyethylene glycol 600 (PEG 600). In certain embodiments, the auxiliary agent is present in an integer amount ranging from 5 wt % to 20 wt %, and the tricalcium silicate is present in an integer amount ranging from 80 wt % to 95 wt %, based on the total weight of the first reagent.

In certain embodiments, the salt material is selected from the group consisting of sodium carbonate, calcium chloride, and a combination thereof.

According to the present disclosure, the water suitable for humans is used to facilitate hardening of the tricalcium silicate of the first reagent and to dissolve the salt material of the second reagent.

According to the present disclosure, the second reagent may further include an auxiliary agent. In certain embodiments, the auxiliary agent is selected from the group consisting of ethanol, glycerol, PEG in liquid form, and combinations thereof. Since the ethanol and the glycerol are similar to the water in terms of having high hydroxyl content, and the PEG in liquid form is hydrophilic, the auxiliary agent may serve as a partial substitute for the aforesaid water or may function as a thickening agent. To be specific, the amount of the water may be reduced by adding the auxiliary agent, or the amount of the water may be maintained even if adding an additional amount of the auxiliary agent. In certain embodiments, the auxiliary agent is present in an integer amount ranging from 5 wt % to 20 wt %, based on the total weight of the second reagent.

In certain embodiments, the salt material is present in an integer amount ranging from 5 wt % to 45 wt %, based on the total weight of the second reagent.

In certain embodiments, the salt material is sodium carbonate that is present in an integer amount ranging from 2 wt % to 45 wt %, based on the total weight of the second reagent. In addition, when the salt material only includes sodium carbonate, the sodium carbonate is present in an amount of not lower than 5 wt %.

In certain embodiments, the salt material is calcium chloride that is present in an integer amount ranging from 2 wt % to 45 wt %, based on the total weight of the second reagent. In addition, when the salt material only includes calcium chloride, the calcium chloride is present in an amount of not lower than 5 wt %.

To be specific, the tricalcium silicate kit does not include calcium carbonate. In other words, the aforesaid auxiliary agents do not include calcium carbonate. The primarily reason is that the presence of the calcium carbonate along with the calcium chloride may result in an extended time period of the hardening process when the first reagent is mixed with the second reagent of the tricalcium silicate kit.

In certain embodiments, a ratio of a volume of the second reagent to a weight of the first reagent in decimal form ranges from 0.2 μL/g to 0.8 μL/g. Packaging the first reagent and the second reagent in an optimal specific ratios (i.e., the aforesaid ratio of the volume of the second reagent to the weight of the first reagent) as the kit ensures that users can effectively mix the first reagent with the second reagent in the desired ratio.

The first embodiment of the tricalcium silicate kit of the present disclosure provides an alternative to existing formulations. Furthermore, the addition of the specific amounts of the sodium carbonate and/or the calcium chloride can shorten a time period of the hardening process, and can even significantly shorten the time period of the hardening process to 2 minutes.

### First Embodiment of Method for Preparing Portland Cement-Based Dental Material

A first embodiment of a method for preparing a Portland cement-based dental material according to the present disclosure includes mixing the first reagent with the second reagent of the first embodiment of the tricalcium silicate kit to undergo a hardening process, so as to form the Portland cement-based dental material. In certain embodiments, a ratio of a volume of the second reagent to a weight of the first reagent in decimal form ranges from 0.2 μL/g to 0.8 μL/g.

The first embodiment of the method for preparing the Portland cement-based dental material provides an alternative to existing methods. In addition, mixing the first reagent with the second reagent in an optimal specific ratio (i.e., the aforesaid ratio of the volume of the second reagent to the weight of the first reagent) can shorten the time period of the hardening process.

### Second Embodiment of Tricalcium Silicate Kit

A second embodiment of a tricalcium silicate kit according to the present disclosure includes a first reagent containing calcium carbonate and tricalcium silicate, and a second reagent containing water.

According to the present disclosure, the first reagent is packaged separately from the second reagent, and the tricalcium silicate kit does not include calcium chloride.

According to the present disclosure, in addition to the tricalcium silicate, the first reagent also includes the calcium carbonate, which shortens the time period of the hardening process when the first reagent is mixed with the second reagent. In certain embodiments, the calcium carbonate is present in an integer amount ranging from 2 wt % to 45 wt %, based on the total weight of the first reagent. Moreover, the first reagent may further include an auxiliary agent. In certain embodiments, the auxiliary agent is selected from the group consisting of ethanol, glycerol, PEG in liquid form, and combinations thereof. In certain embodiments, the auxiliary agent is present in an integer amount ranging from 5 wt % to 20 wt %, based on the total weight of the first reagent.

In certain embodiments, the second reagent may only include the water or may further include a salt material along with the water.

According to the present disclosure, the water suitable for humans is used to facilitate hardening of the tricalcium silicate of the first reagent.

According to the present disclosure, the second reagent may further include an auxiliary agent. In certain embodiments, the auxiliary agent is selected from the group consisting of ethanol, glycerol, PEG in liquid form, and combinations thereof. Since the ethanol and the glycerol are similar to the water in terms of having high hydroxyl content, and the PEG in liquid form is hydrophilic, the auxiliary agent may serve as a partial substitute for the aforesaid water or may function as a thickening agent. To be specific, the amount of the water may be reduced by adding the auxiliary agent, or the amount of the water may be maintained even if adding an additional amount of the auxiliary agent. In certain embodiments, the auxiliary agent is present in an integer amount ranging from 5 wt % to 20 wt %, based on the total weight of the second reagent.

In certain embodiments, the salt material is sodium carbonate that is present in an integer amount ranging from 20 wt % to 45 wt %, based on the total weight of the second reagent.

To be specific, the tricalcium silicate kit does not include calcium chloride. In other words, the aforesaid auxiliary agents do not include calcium chloride. The primarily reason is that the presence of the calcium chloride along with the calcium carbonate may result in an extended time period of the hardening process when the first reagent is mixed with the second reagent of the tricalcium silicate kit.

In certain embodiments, a ratio of a volume of the second reagent to a weight of the first reagent in decimal form ranges from 0.2 μL/g to 0.7 μL/g. Packaging the first reagent and the second reagent in an optimal specific ratio (i.e., the aforesaid ratio of the volume of the second reagent to the weight of the first reagent) as the kit ensures that users can effectively mix the first reagent with the second reagent in the desired ratio.

The second embodiment of the tricalcium silicate kit of the present disclosure provides an alternative to existing formulations. Furthermore, when the tricalcium silicate is mixed with the calcium carbonate, or when the second reagent further includes the specific amounts of the sodium carbonate, the time period of the hardening process can be shortened to not greater than 12 minutes, and even significantly shortened to range from 4 minutes to 8 minutes.

### Second Embodiment of Method for Preparing Portland Cement-Based Dental Material

A second embodiment of a method for preparing a Portland cement-based dental material according to the present disclosure includes mixing the first reagent with the second reagent of the second embodiment of the tricalcium silicate kit to undergo a hardening process, so as to form the Portland cement-based dental material. In certain embodiments, a ratio of a volume of the second reagent to a weight of the first reagent in decimal form ranges from 0.2 μL/g to 0.7 μL/g. When the ratio of the volume of the second reagent to the weight of the first reagent in decimal form is greater than 0.7 μL/g, the time period of the hardening process may be extended.

The second embodiment of the method for preparing the Portland cement-based dental material provides an alternative to existing methods. In addition, mixing the first reagent with the second reagent in an optimal specific ratio (i.e., the aforesaid ratio of the volume of the second reagent to the weight of the first reagent) can maintain or further shorten the time period of the hardening process.

The disclosure will be further described by way of the following examples. However, it should be understood that the following examples are solely intended for the purpose of illustration and should not be construed as limiting the disclosure in practice.

### Examples

### Tricalcium Silicate Kit and Preparation of Portland Cement-Based Dental Material

### Example 1 (EX1)

The materials and the amounts thereof for making the tricalcium silicate kit of EX1 are shown in Table 1 below. As shown in Table 1, the tricalcium silicate kit of EX1 included 0.4 g of a first reagent and 0.25 μL of a second reagent. The first reagent was pure tricalcium silicate (Nanoshel LLC, Cat. no. NS6130-12-001525) with CAS number 12168-85-3. In other words, the tricalcium silicate was present in an amount of 100 wt %, based on the total weight of the first reagent. Sodium carbonate (serving as a salt material, Honeywell, Cat. no. 31432-500G) was present in an amount of 15 wt %, and water was present in an amount of 85 wt %, based on the total weight of the second reagent.

In addition, the Portland cement-based dental material of EX1 was prepared according to the procedures described below. The first reagent and the second reagent of the tricalcium silicate kit of EX1 were mixed to undergo a hardening process, so as to form the Portland cement-based dental material of EX1. A ratio of the volume of the second reagent to the weight of the first reagent in decimal form is 0.63 μL/g.

### Examples 2 to 4 (EX2 to EX4)

The materials and the amounts thereof for making the tricalcium silicate kits of EX2 to EX4 and the procedures for preparing the Portland cement-based dental materials of EX2 to EX4 were similar to those of EX1, except that the weight percentages of the salt material and the water in the second reagent, the volume of the second reagent, and the ratio of the volume of the second reagent to the weight of the first reagent were varied as shown in Table 1 below.

### Examples 5 to 8 (EX5 to EX8)

The materials and the amounts thereof for making the tricalcium silicate kits of EX5 to EX8 and the procedures for preparing the Portland cement-based dental materials of EX5 to EX8 were similar to those of EX1, except that calcium chloride (Shimakyu's Pure Chemicals) was used to replace sodium carbonate, and the weight percentages of the salt material and the water in the second reagent, the volume of the second reagent, and the ratio of the volume of the second reagent to the weight of the first reagent were varied as shown in Tables 1 and 2 below.

### Examples 9 to 12 (EX9 to EX12)

The materials and the amounts thereof for making the tricalcium silicate kits of EX9 to EX12 and the procedures for preparing the Portland cement-based dental materials of EX9 to EX12 were similar to those of EX1, except that the salt material included both the sodium carbonate and the calcium chloride, and the weight percentages of the salt material and the water in the second reagent, the volume of the second reagent, and the ratio of the volume of the second reagent to the weight of the first reagent were varied as shown in Table 2 below.

A respective one of the Portland cement-based dental materials of EX1 to EX12 was prepared using the first embodiment of the tricalcium silicate kit of the present disclosure as mentioned above. The detailed information of the tricalcium silicate kits used for preparing the Portland cement-based dental materials of EX1 to EX12 is summarized in Tables 1 and 2.

TABLE 1

| | | | | EX1 | EX2 | EX3 | EX4 | EX5 | EX6 |
|---|---|---|---|---|---|---|---|---|---|
| First | Tricalcium | | wt | 100 | 100 | 100 | 100 | 100 | 100 |
| reagent | silicate | | % | | | | | | |
| | Weight of first | | g | 0.4 | 0.4 | 0.4 | 0.4 | 0.4 | 0.4 |
| | reagent | | | | | | | | |
| Second | Salt | Sodium | wt | 15 | 15 | 20 | 20 | | |
| reagent | ma- | carbonate | % | | | | | | |
| | terial | Calcium | wt | | | | | 15 | 15 |
| | | chloride | % | | | | | | |
| | | Total | wt | 15 | 15 | 20 | 20 | 15 | 15 |
| | | amount | % | | | | | | |
| | | of sodium | | | | | | | |
| | | carbonate | | | | | | | |
| | | and | | | | | | | |
| | | calcium | | | | | | | |
| | | chloride | | | | | | | |

| | | | | | | | |
|---|---|---|---|---|---|---|---|
| Water | wt | 85 | 85 | 80 | 80 | 85 | 85 |
| | % | | | | | | |
| Total amount | wt | 100 | 100 | 100 | 100 | 100 | 100 |
| ofsalt material | % | | | | | | |
| and water | | | | | | | |
| Volume of | μL | 0.25 | 0.31 | 0.25 | 0.31 | 0.21 | 0.25 |
| second reagent | | | | | | | |

| | | | | | | |
|---|---|---|---|---|---|---|
| Ratio of volume of second | 0.63 | 0.78 | 0.63 | 0.78 | 0.53 | 0.63 |
| reagent to weight of first | | | | | | |
| reagent (in decimal form) | | | | | | |

TABLE 2

| | | | | EX | EX | EX | EX | EX | EX |
|---|---|---|---|---|---|---|---|---|---|
| | | | | 7 | 8 | 9 | 10 | 11 | 12 |
| First | Tricalcium | | wt | 100 | 100 | 100 | 100 | 100 | 100 |
| reagent | silicate | | % | | | | | | |
| | Weight of | | g | 0.4 | 0.4 | 0.4 | 0.4 | 0.4 | 0.4 |
| | first reagent | | | | | | | | |
| Second | Salt | Sodium | wt | | | 15 | 15 | 15 | 20 |
| reagent | ma- | carbonate | % | | | | | | |
| | terial | Calcium | wt | 20 | 20 | 15 | 15 | 20 | 15 |
| | | chloride | % | | | | | | |
| | | Total | wt | 20 | 20 | 30 | 30 | 35 | 35 |
| | | amount of | % | | | | | | |
| | | sodium | | | | | | | |
| | | carbonate | | | | | | | |
| | | and | | | | | | | |
| | | calcium | | | | | | | |
| | | chloride | | | | | | | |

| | | | | | | | |
|---|---|---|---|---|---|---|---|
| Water | wt | 80 | 80 | 70 | 70 | 65 | 65 |
| | % | | | | | | |
| Total amount of | wt | 100 | 100 | 100 | 100 | 100 | 100 |
| salt material | % | | | | | | |
| and water | | | | | | | |
| Volume of | μL | 0.21 | 0.25 | 0.21 | 0.25 | 0.21 | 0.21 |
| second reagent | | | | | | | |

| | | | | | | |
|---|---|---|---|---|---|---|
| Ratio of volume of second | 0.53 | 0.63 | 0.53 | 0.63 | 0.53 | 0.53 |
| reagent to weight of first | | | | | | |
| reagent (in decimal form) | | | | | | |

### Examples 13 to 15 (EX13 to EX15)

The materials and the amounts thereof for making the tricalcium silicate kits of EX13 to EX15 and the procedures for preparing the Portland cement-based dental materials of EX13 to EX15 were similar to those of EX1, except that the second reagent included only the water and did not include the salt material, the first reagent further included calcium carbonate (Emperor Chemical Co. LTD) in addition to the tricalcium silicate, and the amount of the calcium carbonate in the first reagent, and the amount of the water in the second reagent were varied as shown in Table 3 below. In particular, in the respective one of the first reagents of EX13 to EX15, the calcium carbonate was present in an amount ranging from 10 wt % to 30 wt %, based on the total weight of the first reagent.

### Examples 16 and 17 (EX16 and EX17)

The materials and the amounts thereof for making the tricalcium silicate kits of EX16 and EX17 and the procedures for preparing the Portland cement-based dental materials of EX16 and EX17 were similar to those of EX14, except that the second reagents further included the sodium carbonate serving as the salt material in addition to the water, and the volume of the second reagent, and the ratio of the volume of the second reagent to the weight of the first reagent were varied as shown in Table 3 below. In particular, in the respective one of the second reagents of EX16 and EX17, the sodium carbonate was present in an amount of 20 wt %, based on the total weight of the second reagent.

A respective one of the Portland cement-based dental materials of EX13 to EX17 was prepared using the second embodiment of the tricalcium silicate kit of the present disclosure as mentioned above. The detailed information of the tricalcium silicate kits used for preparing the Portland cement-based dental materials of EX13 to EX17 is summarized in Table 3.

TABLE 3

| | | | | EX13 | EX14 | EX15 | EX16 | EX17 |
|---|---|---|---|---|---|---|---|---|
| First | Tricalcium | | wt | 90 | 80 | 70 | 80 | 80 |
| reagent | silicate | | % | | | | | |
| | Calcium | | wt | 10 | 20 | 30 | 20 | 20 |
| | carbonate | | % | | | | | |
| | Total amount | | wt | 100 | 100 | 100 | 100 | 100 |
| | of tricalcium | | % | | | | | |
| | silicate | | | | | | | |
| | and calcium | | | | | | | |
| | carbonate | | | | | | | |
| | Weight of first | | g | 0.4 | 0.4 | 0.4 | 0.4 | 0.4 |
| | reagent | | | | | | | |
| Second | Salt | Sodium | wt | | | | 20 | 20 |
| reagent | ma- | carbonate | % | | | | | |
| | terial | Calcium | wt | | | | | |
| | | chloride | % | | | | | |
| | | Total | wt | | | | 20 | 20 |
| | | amount | % | | | | | |
| | | of | | | | | | |
| | | sodium | | | | | | |
| | | carbonate | | | | | | |
| | | and | | | | | | |
| | | calcium | | | | | | |
| | | chloride | | | | | | |

| | | | | | | |
|---|---|---|---|---|---|---|
| Water | wt | 100 | 100 | 100 | 80 | 80 |
| | % | | | | | |
| Total amount | wt | 100 | 100 | 100 | 100 | 100 |
| of salt | % | | | | | |
| material | | | | | | |
| and water | | | | | | |
| Volume of | μL | 0.25 | 0.25 | 0.25 | 0.21 | 0.25 |
| second reagent | | | | | | |

| | | | | | |
|---|---|---|---|---|---|
| Ratio of volume of second | 0.63 | 0.63 | 0.63 | 0.53 | 0.63 |
| reagent to weight of first | | | | | |
| reagent (in decimal form) | | | | | |

### Comparative Example 1 (CE1)

The materials and the amounts thereof for making the tricalcium silicate kit of CE1 are shown in Table 4 below, and the Portland cement-based dental material of CE1 were prepared by directly mixing 0.4 g of tricalcium silicate with 0.25 μL of water.

### Comparative Example 2 (CE2)

The materials and the amounts thereof for making the tricalcium silicate kit of CE2 and the procedures for preparing the Portland cement-based dental material of CE2 were similar to those of EX16 and CE17, except that the volume of the second reagent, and the ratio of the volume of the second reagent to the weight of the first reagent were varied as shown in Table 4 below. In particular, the ratio of the volume of the second reagent to the weight of the first reagent was increased from 0.53 in EX16 and 0.63 in EX17 to 0.78 in CE2.

### Comparative Examples 3 to 7 (CE3 to CE7)

The materials and the amounts thereof for making the tricalcium silicate kits of CE3 to CE7 and the procedures for preparing the Portland cement-based dental materials of CE3 to CE7 were similar to those of EX14, except that a respective one of the second reagents of CE3 to CE7 included calcium chloride (serving as a salt material), a respective one of the second reagents of CE6 and CE7 further included sodium carbonate (serving as a salt material) in addition to calcium chloride, and the volume of the second reagent, and the ratio of the volume of the second reagent to the weight of the first reagent were varied as shown in Table 4 below.

TABLE 4

| | | | | CE1 | CE2 | CE3 | CE4 | CE5 | CE6 | CE7 |
|---|---|---|---|---|---|---|---|---|---|---|
| First | Tricalcium | | wt | 100 | 80 | 80 | 80 | 80 | 80 | 80 |
| reagent | silicate | | % | | | | | | | |
| | Calcium | | wt | | 20 | 20 | 20 | 20 | 20 | 20 |
| | carbonate | | % | | | | | | | |
| | Total amount | | wt | 100 | 100 | 100 | 100 | 100 | 100 | 100 |
| | of tricalcium | | % | | | | | | | |
| | silicate | | | | | | | | | |
| | and calcium | | | | | | | | | |
| | carbonate | | | | | | | | | |
| | Weight of first | | g | 0.4 | 0.4 | 0.4 | 0.4 | 0.4 | 0.4 | 0.4 |
| | reagent | | | | | | | | | |
| Second | Salt | Sodium | wt | | 20 | | | | 15 | 20 |
| reagent | ma- | carbonate | % | | | | | | | |
| | terial | Calcium | wt | | | 20 | 20 | 20 | 15 | 20 |
| | | chloride | % | | | | | | | |
| | | Total | wt | 0 | 20 | 20 | 20 | 20 | 30 | 40 |
| | | amount of | % | | | | | | | |
| | | sodium | | | | | | | | |
| | | carbonate | | | | | | | | |
| | | and | | | | | | | | |
| | | calcium | | | | | | | | |
| | | chloride | | | | | | | | |

| | | | | | | | | |
|---|---|---|---|---|---|---|---|---|
| Water | wt | 100 | 80 | 80 | 80 | 80 | 70 | 60 |
| | % | | | | | | | |
| Total amount of | wt | 100 | 100 | 100 | 100 | 100 | 100 | 100 |
| salt material | % | | | | | | | |
| and water | | | | | | | | |
| Volume of | μL | 0.25 | 0.31 | 0.21 | 0.25 | 0.31 | 0.25 | 0.25 |
| second reagent | | | | | | | | |

| | | | | | | | |
|---|---|---|---|---|---|---|---|
| Ratio of volume of second | 0.63 | 0.78 | 0.53 | 0.63 | 0.78 | 0.63 | 0.63 |
| reagent to weight of first | | | | | | | |
| reagent (in decimal form) | | | | | | | |

### Property Evaluation

### A. Measurement of Hardening Time

Each of the Portland cement-based dental materials of EX1 to EX17 and CE1 to CE7 served as a test specimen, and the time period for hardening of the test specimen was determined using a Gilmore needle apparatus (Gilson Company, Inc.). To be specific, the test specimen was struck once per minute with a free falling Gilmore needle of the Gilmore needle apparatus. The time period for hardening refers to the time point when the same location on the surface of the test specimen was struck until the indentation resulting from the previous strike(s) on the surface no longer deepened, or when different locations on the surface of the test specimen were struck until the surface no longer showed a new indentation. The results are shown in Table 5 below.

### Results

TABLE 5

| | EX1 | EX2 | EX3 | EX4 | EX5 | EX6 |
|---|---|---|---|---|---|---|
| Time period for | 4 | 4 | 4 | 4 | 4 | 8 |
| hardening (min) | | | | | | |
| | EX7 | EX8 | EX9 | EX10 | EX11 | EX12 |
| Time period for | 4 | 8 | 4 | 8 | 2 | 4 |
| hardening (min) | | | | | | |
| | EX13 | EX14 | EX15 | EX16 | EX17 | CE1 |
| Time period for | 12 | 8 | 8 | 4 | 10 | 12-14 |
| hardening (min) | | | | | | |
| | CE2 | CE3 | CE4 | CE5 | CE6 | CE7 |
| Time period for | >14 | >14 | >14 | >14 | >14 | >14 |
| hardening (min) | | | | | | |

Referring to Table 5, the time period for hardening determined in each of the test specimens of EX1 to EX12 was shorter than that determined in the test specimen of CE1. Specifically, the time period for hardening determined in EX1 to EX10 and EX12 was shorten to 4 to 8 minutes, and the time period for hardening determined in EX11 was further shorten to 2 minutes. These results indicate that, by virtue of the combination of the tricalcium silicate, the water and the salt material including the sodium carbonate and/or the calcium chloride, each of the tricalcium silicate kits of EX1 to EX12 can effectively shorten the time period for hardening of the Portland cement-based dental material.

Referring back to Table 5, the time period for hardening determined in each of the test specimens of EX13 to EX15 may be equal to or shorter than that determined in the test specimen of CE1. Specifically, by virtue of the first reagent including 10% to 30% of the calcium carbonate and the tricalcium silicate, even though the second reagent only included water without the salt material, the time period for hardening determined in each of the test specimens of EX13 to EX15 was not greater than 12 minutes. Moreover, when the first reagent included 20% to 30% of the calcium carbonate and the tricalcium silicate (i.e., the tricalcium silicate kits of EX14 and EX15), the time period for hardening determined in each of the test specimens of EX14 and EX15 was further shortened to 8 minutes, which was two-thirds of the time period for hardening determined in CE1 (i.e., 12 minutes). These results demonstrate that each of the tricalcium silicate kits of EX13 to EX15 can maintain or effectively shorten the time period for hardening of the Portland cement-based dental material.

Referring to Table 5 again, the time period for hardening determined in each of the test specimens of EX16 and EX17 was shorter than that determined in the test specimen of CE1, and the time period for hardening determined in the test specimen of CE2 was longer than that determined in the test specimen of CE1. Specifically, by virtue of the first reagent including an appropriate amount of the calcium carbonate in combination with the ratio of the volume of the second reagent to the weight of the first reagent in decimal form ranging from 0.53 μL/g to 0.63 μL/g, the time period for hardening of each of the test specimens of EX16 and EX17 was not greater than 12 minutes, and was even further shortened to range from 4 minutes to 10 minutes. However, when the ratio of the volume of the second reagent to the weight of the first reagent in decimal form was 0.78 μL/g (i.e., the tricalcium silicate kit of CE2), the time period for hardening of the test specimen was greater than 14 minutes. These results indicate that when the ratio of the volume of the second reagent to the weight of the first reagent was limited to be lower than 0.65 μL/g (i.e., the respective one of the tricalcium silicate kits of EX16 and EX17), the time period for hardening of the Portland cement-based dental material can be effectively shortened.

Referring back to Table 5, the time period for hardening determined in each of the test specimens of CE3 to CE7 was longer than that determined in the test specimen of CE1. Specially, for the respective one of tricalcium silicate kits of CE3 to CE7, when the first reagent included the calcium carbonate and the second reagent included the calcium chloride, the time period for hardening time of the test specimen was greater than 14 minutes. These results demonstrate that the presence of both the calcium carbonate and the calcium chloride in the tricalcium silicate kit may result in an extended time period for hardening of the Portland cement-based dental material.

Summarizing the above test results, it is clear that when the first embodiment of the tricalcium silicate kit (i.e., EX1 to EX12) or the second embodiment of the tricalcium silicate kit (i.e., EX13 to EX17) of the present disclosure was used for preparing the Portland cement-based dental material, the time period for hardening of such Portland cement-based dental material may be effectively shortened.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A tricalcium silicate kit, comprising:
a first reagent which includes tricalcium silicate; and
a second reagent which includes a salt material and water, the salt material being present in an amount ranging from 5 wt % to 45 wt %, based on the total weight of the second reagent;
wherein the salt material is selected from the group consisting of sodium carbonate, calcium chloride, and a combination thereof.

2. The tricalcium silicate kit according to claim 1, wherein a ratio of a volume of the second reagent to a weight of the first reagent in decimal form ranges from 0.2 μL/g to 0.8 μL/g.

3. A method for preparing a Portland cement-based dental material, comprising
mixing a first reagent with a second reagent of a tricalcium silicate kit as claimed in claim 1 to undergo a hardening process, so as to form the Portland cement-based dental material,
wherein a ratio of a volume of the second reagent to a weight of the first reagent in decimal form ranges from 0.2 μL/g to 0.8 μL/g.

4. A tricalcium silicate kit, comprising:
a first reagent which includes calcium carbonate and tricalcium silicate, the calcium carbonate being present in an amount ranging from 2 wt % to 45 wt %, based on the total weight of the first reagent; and
a second reagent which includes water.

5. The tricalcium silicate kit according to claim 4, wherein the second reagent further includes sodium carbonate serving as a salt material, the sodium carbonate being present in an amount ranging from 20 wt % to 45 wt %, based on the total weight of the second reagent.

6. The tricalcium silicate kit according to claim 4, wherein a ratio of a volume of the second reagent to a weight of the first reagent in decimal form ranges from 0.2 μL/g to 0.7 μL/g.

7. A method for preparing a Portland cement-based dental material, comprising
mixing a first reagent with a second reagent of a tricalcium silicate kit as claimed in claim 4 to undergo a hardening process, so as to form the Portland cement-based dental material,
wherein a ratio of a volume of the second reagent to a weight of the first reagent in decimal form ranges from 0.2 μL/g to 0.7 μL/g.
